(19) United States
(12) Patent Application Publication
(10) Pub. No.: US 2007/0248210 A1
(43) Pub. Date: Oct. 25, 2007

(54) Automatic Positioning Quality Assessment for Digital Mammography

(76) Inventors: Emil Selse, Linkopong (SE); Kristina Pettersson, Linkoping (SE)

Correspondence Address: YOUNG & THOMPSON, 745 SOUTH 23RD STREET, 2ND FLOOR, ARLINGTON, VA, 22202, US

(21) Appl. No.: 10/571,789

(22) PCT Filed: Sep. 22, 2003
(86) PCT No.: PCT/SE03/01477

§ 371 Date: May 24, 2007

(51) Int. Cl. A61B 6/04; G06T 7/00

(52) U.S. Cl. 378/37; 382/132

(57) ABSTRACT

The invention presents a way to automatically assess the quality of acquired (S1, S2) digital mammographic images with respect to the image positioning of a patient's breast. The automated digital quality assessment (S3) is executed in real time and preferably notifies (S4) the technologist instantly if the image positioning quality of a mammographic image is inadequate. This makes it possible to retake the image while the patient is still present at the examination facility. The quality assessment notification includes information to the technologist, preferably both visually and statistically of land-mark positioning measurements. Alternatively, the digital quality assessment is accompanied by a computerized decision of whether the mammogram needs to be retaken, requiring a minimum of involvement, if at all, from the technologist. The invention hence provides a set of quality-assured mammographic images that can be stored (S5) and later accessed by a radiologist for review and diagnosis (S6).

### TECHNICAL FIELD OF THE INVENTION

The present invention generally relates to the field of radiology and mammography, and more specifically to assessment of positioning image quality in digital mammography.

### BACKGROUND OF THE INVENTION

Mammography is today the most important and accurate method for diagnosing breast cancer. The diagnostic value of mammography images is highly dependent on image quality, which, in turn, depends on a number of technical and image processing related factors such as proper exposure, compression, sharpness and contrast. However, if the breast is improperly positioned the radiologist might not be able to evaluate the mammogram correctly. With digital mammography, parameters such as brightness, contrast, magnification etc. can be altered in real time after the mammography examination is completed to improve recognition of suspicious lesions in the breast. Different types of segmentations can also be applied to distinguish certain areas such as the nipple and the skin-line, as outlined in reference [1].

Several theoretical claims regarding optimal mammography image positioning are established by various authorities in this field. The vertical length of the pectoral muscle, inclusion of the whole breast, visibility of the area just below the breast and a tangentially placed nipple are some examples of important criteria. These criteria are well covered and summarized in references [2, 3] by Eklund, G. W. Cardenosa. These references describe characteristics of a “good” mammogram together with a description of the important landmarks mentioned above. General mammography quality factors can be found in the European Guidelines, recommended by the European commission [4].

In reference [5], the authors Edström and Ståhl have prepared criteria of quality assessment for film-based mammography as a base for Swedish guidelines, in order to get quality-assured imaging at mammography screening. They performed a quality control where important landmarks on 1194 mammograms were measured manually in order to evaluate the image quality. However these kinds of measurements are done manually and thereby time consuming and are unlikely to be performed on a daily basis, especially if one considers the amount of time generally available for each mammography examination at a normal mammography unit. Even more time consuming is the procedure with conventional film-based mammography where the images have to be developed and hanged on a light-box before an assessment of the image quality can be determined. This generally means that the patient has already left the examination facility before the image quality is secured, and that the patient might have to be recalled in case the images were not good enough. It is also likely to believe that situations occur where mediocre images are accepted for diagnosis, since the woman will be invited for a new mammography examination within a certain amount of time anyway. Finally, in addition to the administrative work for recalling the patient, and the workload for the staff to start a new examination from the beginning again, it is also reasonable to believe that being recalled might cause anxiety for the woman.

### SUMMARY OF THE INVENTION

The present invention overcomes these and other drawbacks of the prior art.

It is a general object to provide an improved method, system and software for mammographic image quality assurance. In this respect, it is especially important to provide automatic or automated quality assessment of a digital mammographic image with respect to the positioning of a patient's breast.

It is particularly desirable to be able to assess whether the acquired mammogram is good enough for satisfactory reviewing immediately after image acquisition, before the woman leaves the examination. The invention should thus preferably serve as a support for making decisions with respect to the adequacy of acquired mammograms.

It is also a main object of the invention to improve the overall mammography process and reduce the number of recalls due to insufficiently positioned mammographic images.

Yet another object of the invention, in the long run, is to facilitate a common standardization of mammography image quality and thereby simplify the work of the technologists.

Still another object of the invention is to handle one or several different projections and also manage several retakes of mammography images due to indications of insufficiently positioned images, given from the invention.

These and other objects are met by the invention as defined by the accompanying patent claims.

The basic idea according to the invention is to automatically secure mammographic image positioning quality in real time. The invention basically involves computerized algorithmic processing of one or more digital mammographic images of a patient based on at least one predetermined criterion for image positioning with respect to the breast for assessing the image positioning quality. A positioning quality assessment result is produced in real-time based on the computerized algorithmic processing, and it is then determined whether a mammographic image needs to be retaken with improved positioning. This opens up for immediate image retaking, if this is necessary, while the patient is still present at the examination facility, and eliminates or at least reduces the need for recalling the patient.

The positioning quality assessment result is preferably communicated immediately to the technologist on a user interface to enable a real-time decision to retake the mammographic image. The positioning quality assessment result typically includes a number of user-configurable parameters, thus allowing a selectable level of acceptance considering the breast positioning quality and also a selectable level of detail in communicating the result.

The image positioning quality result preferably comprises a visual result and/or a statistics part. The visual part informs the technologist of inadequately positioned parts of the mammographic image by highlighting these areas, and the statistics gives the technologist an assessment of the image based on threshold values for criteria of a well-positioned image.

Alternatively, the positioning quality system of the invention is fully automated with a minimum of involvement, if at all, from the technologist. In the case of a fully automated system, a computerized decision of whether the mammogram needs to be retaken is made based on the quality assessment result. If desired, this decision may be checked by a technologist before the retake is effectuated, giving the technologist a chance to abort another round of X-ray exposure of the breast.

If required or otherwise desired, the invention can handle several different projections, including MLO (Medio-lateral oblique), CC (Cranio-caudal), LM (Latero-medial) and ML (Medio-lateral) projections as well as combinations thereof. The invention is also capable of managing several retakes of mammographic images due to indications of insufficiently positioned images, continuously updating which image or set of images among the retakes that is considered most adequate.

Although the invention is especially suitable for mammographic screening, it can also be applied in clinical mammography.

The invention offers the following advantages:
Improved overall mammography process;
Automated, real-time quality assessment of a digital mammographic image with respect to the positioning of a patient's breast.
Reduced number of recalls due to insufficiently positioned mammographic images.
Facilitates a common standardization of mammography image quality and simplifies the work of the technologists.
Automated control of the accuracy of the breast positioning compensates for technologists with inadequate education concerning breast positioning, and also makes the image acquisition more effective because indications of insufficient positioning can be made instantly.

Other advantages offered by the present invention will be appreciated upon reading of the below description of the embodiments of the invention.

### BRIEF DESCRIPTION OF THE DRAWINGS

The invention, together with further objects and advantages thereof, will be best understood by reference to the following description taken together with the accompanying drawings, in which:

FIG. 1 is a schematic diagram of an exemplary computer-aided system for generating and processing mammograms together with the invention;

FIG. 2 illustrates a general exemplary mammography workflow with the invention included as the dotted parts;

FIG. 3 is a flow chart of an exemplary mammography image acquisition workflow on a more detailed level;

FIGS. 4A and 4B illustrate important landmarks on mammograms of MLO (Medio-lateral oblique) and CC (Cranio-caudal) projections, respectively; and

FIGS. 5A and 5B schematically illustrate examples of visual indication of inadequately positioned parts of a breast on mammograms of MLO and CC projections, respectively.

### DETAILED DESCRIPTION OF EMBODIMENTS OF THE INVENTION

Throughout the drawings, the same reference characters will be used for corresponding or similar elements.

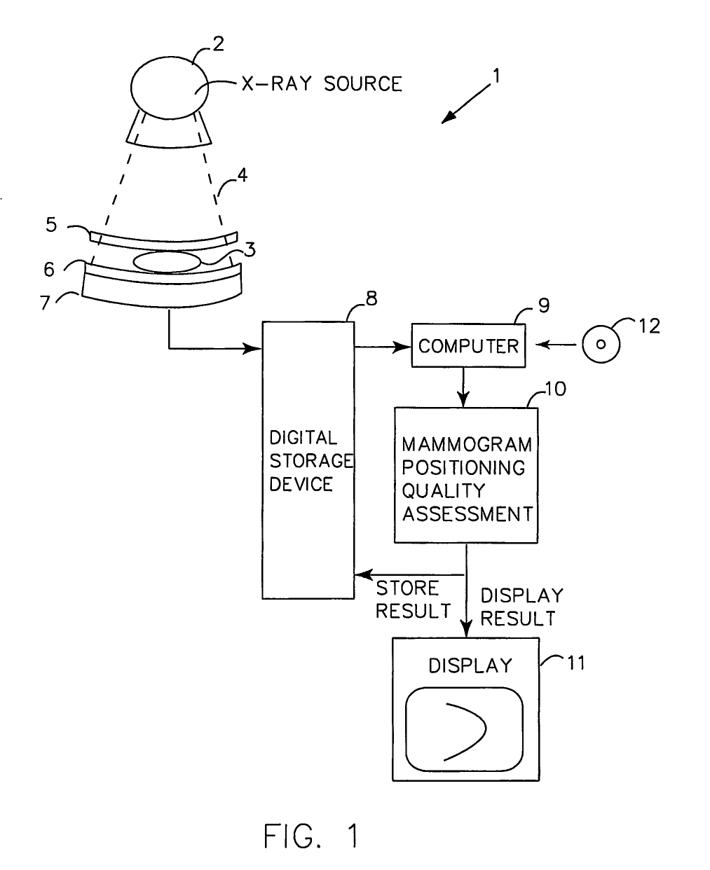

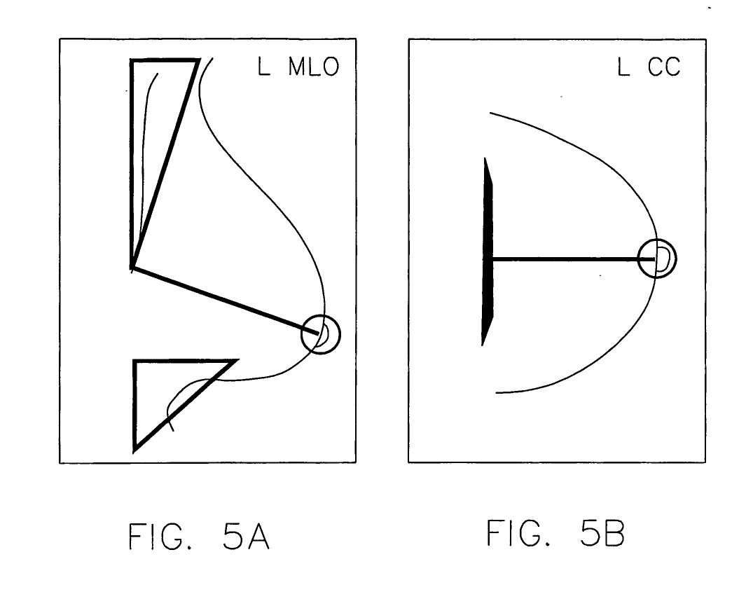

For a better understanding of the invention, it may be useful to begin with a general system overview of an exemplary digital mammography system, referring to FIG. 1. The system 1 includes an X-ray source 2 directed to expose a patient's breast 3 with X-ray beams 4. The breast is generally compressed, using a predefined compression force, between two compression plates 5, 6. Below the lower compression plate, some detector means 7 is arranged. The detector 7 is usually in the form of a two-dimensional array of radiation sensitive elements, the outputs of which are mapped into a corresponding array of digital pixels representing the digital mammogram. The digital pixel information is stored as an image file in a digital storage device 8, and the digital mammogram is then generated in a computer-based acquisition workstation, for example the Sectra Acquisition workstation within the Sectra MicroDose Mammography System 9. The Sectra acquisition workstation software has tight RIS (Radiology Information System) integration and is DICOM (Digital Imaging and Communications in Medicine) compatible for transfer of images to a storage device, for example Sectra PACS (Picture Archiving and Communication System) and reviewing stations. In accordance with the invention, the system also comprises a system module 10 for mammogram positioning quality assessment in order to control the quality and accuracy of the image with respect to the positioning of the breast. Instantly when the digital image file is created within the acquisition workstation, the image processing algorithms of the quality assessment system 10 of the invention are executed, normally without any user intervention, and then preferably displayed for the user on a display device 11. The image is typically displayed in the application window of the acquisition workstation. If the result of the quality assessment indicates inadequate positioning of the mammogram, a preferred feature of the invention is to clearly highlight inadequate areas on the displayed image. For example this can be achieved by displaying a simple outline of a geometric object as an overlay on top of the mammogram image, like a rectangle or circle, around the area of concern, as exemplified in FIGS. 5A and B. Finally the results of the quality assessment are preferably stored together with the image for easy availability later.

Advantageously, the quality assessment module 10 is implemented as a software module that operates integrated with existing software for an image acquisition workstation of a digital mammography system. The actual software code may initially be provided on any type of computer-readable medium 12 for subsequent installation in executable form as a quality assessment module 10 on the computer-based acquisition workstation 9.

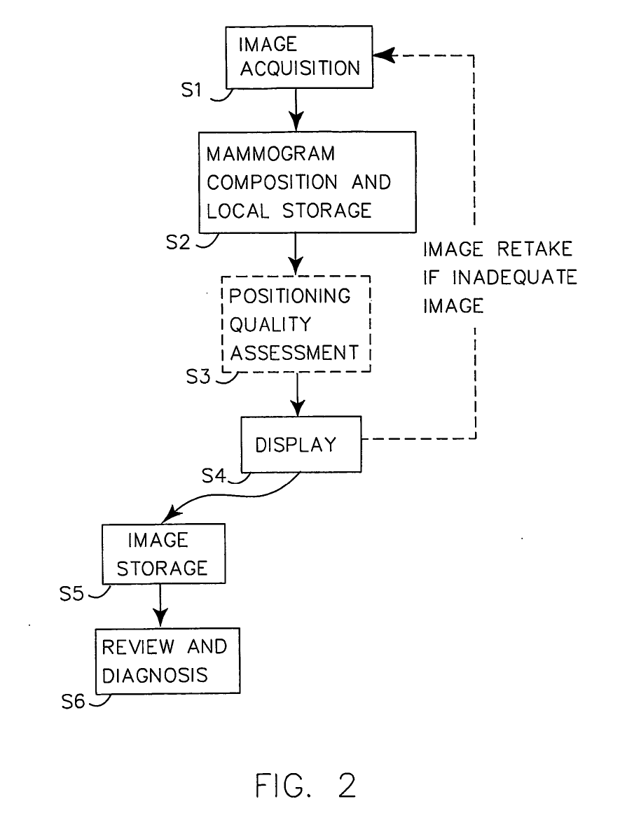

At this point it may be useful to explain the difference between a general digital mammography workflow with and without the invention included, referring to the exemplary workflow diagram of FIG. 2. In the diagram of FIG. 2, the dotted parts represent the outline of the invention. The workflow starts with the actual mammography examination and image acquisition S1 using the normal mammography stand, which is connected to a computer in which the actual mammogram image is composed and temporarily stored S2. The next step, S3, illustrates a main difference between a general workflow of today and a workflow where the image positioning quality is controlled. The quality assessment, which is based on computerized, algorithmic processing of acquired digital mammography images, gives the technologist a guarantee that at least one mammographic image of satisfactory quality will be produced before the patient leaves the examination. Preferably, maximum breast tissue should be included in an optimally positioned mammogram, clearly visualizing the whole breast. If the image have parts, which are not sufficiently positioned, those parts may be marked when the image is displayed on the screen S4. In such a case, the image is preferably retaken and the mammographic quality of the retaken image is controlled as well, until a mammogram with adequate breast positioning is finally acquired. When finishing the examination, images of a predefined set of projections are successfully obtained and quality assured. Generally, the mammograms are then sent to a storing device, PACS, S5 and accessed by the radiologist for review and diagnosis S6.

In the prior art, the step of controlling the positioning quality is not automated, and not even regularly performed manually at every mammography examination. This means that there is no guarantee that the mammograms are good enough for adequate diagnosis. Especially if one considers that most often there is a great amount of mammograms acquired every day at a normal mammography unit, which makes it hard to keep a sufficiently high and consistent quality. The invention on the other hand provides a remedy to these problems, and delivers a breast positioning quality assessment in real-time based on computerized algorithmic processing of the mammographic images. Based on the positioning quality assessment result, it is then determined (more or less instantly), either by a technologist or as part of a fully automated quality-assurance procedure, whether a mammographic image needs to be retaken with improved positioning. This makes it possible to retake images while the patient is still present at the examination facility, and thus significantly reduces the need for recalling the patient.

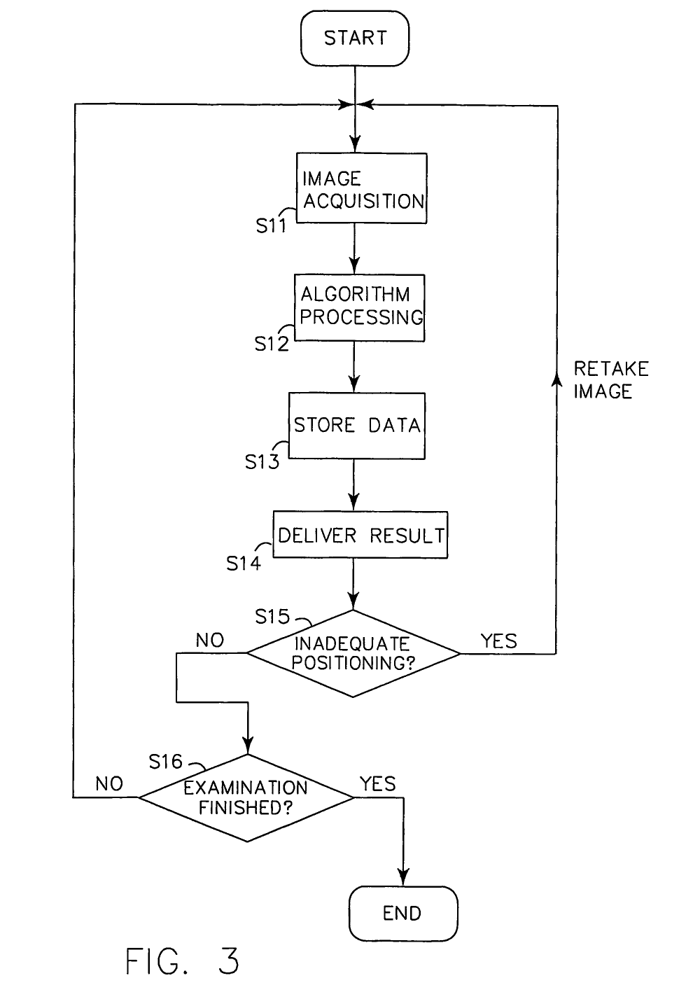

FIG. 3 illustrates an exemplary mammography image acquisition workflow on a more detailed level, where the basic steps from the beginning to the end of the examination are covered. In step S11, examination is performed and one or more mammographic images are acquired. Step S12 involves algorithmic image processing based on at least one predetermined criterion for optimal image positioning with respect to the breast, as will be exemplified later on. The algorithms, which constitute the basis of the mammographic positioning quality control module, are preferably part of the software for an acquisition workstation as a built-in functionality or plug-in. The algorithms are normally executed instantly after image acquisition. Preferably, the quality assessment results are locally stored in step S13. In a semi-automated quality assessment system, the quality assessment results are delivered to the technologist S14, which makes the fmal decision as to whether or not the breast is properly positioned S15. In a fully automated system, a computerized decision is made based on the results of the algorithmic processing previously performed in step S12. The quality assessment results are normally produced based on image processing of important landmarks together with a set of predefined threshold values. If it is determined, either by the technologist or in a fully automated manner, that the breast positioning is inadequate, the image is retaken (preferably during the same examination) and another round of image acquisition and quality control is initiated. Before image retaking, the patient is typically repositioned in dependence on the quality assessment result, and in particular in dependence on the visual indication of inadequately positioned parts of the breast. In a fully automated system, however, it may be possible to adjust the positioning of the X-ray tube and/or image detector in order to improve the positioning, using a control signal feedback from the quality assessment module to the movable X-ray tube and/or detector. When a set of good images are finally acquired the examination S16 is finished and the images are sent to a storage device and are ready for review and diagnosis.

Digital mammography opens up for new applications in the image processing area and allows the technologist to evaluate mammograms quickly for correct positioning and assess exclusion of artifacts immediately, before the woman leaves the examination. This increases the ability of obtaining higher quality of mammography images. In order to increase the number of detected lesions in mammograms, it is of great importance that optimal amount of tissue is included for adequate visualization of areas of clinical concern. Previously, it has been necessary to use only experienced technologists properly educated in the field of breast-positioning for mammography image acquisition in order to provide adequate visualization. Still, human mistakes occur, and poorly positioned mammograms may lead to incorrect diagnosis, especially if lesions are missed. The automated quality assurance system of the invention however effectively prevents poorly positioned images from constituting the basis for review and diagnosis, since new mammograms are always acquired when the quality assessment functionality indicates that retaking is necessary. In this way, a complete and adequate set of images is given to the radiologist for review and diagnosis.

Furthermore, it is reasonable to believe that an automatic quality control system might improve the technologist's knowledge of image positioning quality and increase the concentration since the technologist is notified every time a mammogram is insufficiently positioned and can therefore learn how to position the breast correctly.

The algorithms in the quality assessment method are preferably focused on processing areas that are considered important landmarks. From the outcome of checking such areas, an assessment is produced indicating whether the breast is optimally positioned and if the whole breast is visualized or not.

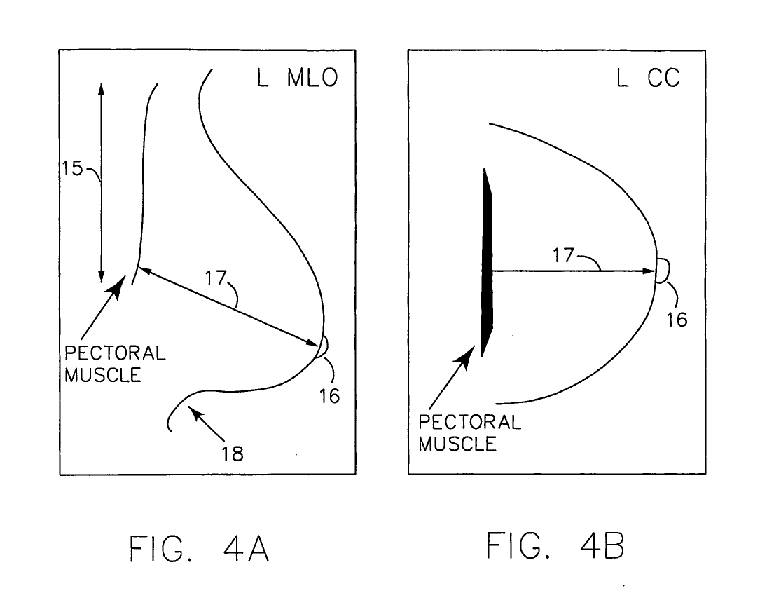

Referring to FIG. 4A, the most important landmarks on an MLO (Medio-lateral oblique) mammogram comprises adequate vertical length 15 of the pectoral muscle, a tangentially positioned nipple 16, the difference between a CC and MLO posterior nipple line 17 within accepted limits, and a clearly visible inframammary fold 18. The inframammary fold is situated below the glandular tissue and shall be clearly visible, which indicates that the whole breast is included on the image. The requirement for a tangential nipple 16 is requested for mammograms with CC projection as well. Normally, it is sufficient if the nipple is positioned tangentially on either the CC or MLO mammogram (when performing standard screening.) The posterior nipple line (PNL) on an MLO mammogram is defined as the distance from the nipple skin-junction to the pectoral muscle, or to the back of the image if the muscle is too short. The same distance 17 is measured on the CC mammogram (see FIG. 4B) of the same breast, where the distance is measured to the back of the image, regardless of whether the pectoral muscle is visible or not. The difference between the two measurements should be within a certain limit (normally about 15 millimeters.)

The initial step for identifying the pectoral muscle comprises basic thresholding to segment the breast from the background. Next, an ROI (Region of Interest) image of the pectoral muscle area is created and then sliced further into thin stripes, which simplifies identification of pectoral muscles with a non-straight shape. Each stripe is pre-processed where the main step includes calculating a thresholded gradient image, before the Hough transform is applied on each stripe. The outcome is a set of points along the edge of the pectoral muscle. The points are interpolated using a third degree curve to give a smooth curve along the muscle edge. The vertical distance is then easily measured.

The visibility of the inframammary fold is determined by calculating the derivative, at regular short intervals, along the skin-line in the area close to the chest wall. To determine whether the nipple is tangentially positioned or not a breast skin edge-detection algorithm is used together with analysis of the gradients. The measurements of the posterior nipple lines on MLO (FIG. 4A) and CC (FIG. 4B) mammograms are accomplished using the prior information about the positions of the nipple and pectoral muscle. It should be understood that a whole set of different conventional algorithms for performing the necessary image processing and measurements on the breast are available for use by the skilled person. Other algorithms than those specifically mentioned above could be used by the invention. As long as the algorithms serve the same overall purpose, they can be designed differently and use other image processing techniques.

The above algorithm(s), steps and actions are preferably implemented as computer program elements such as functions, procedures or equivalents. These program elements may be written in a functional programming language, an object oriented programming language or any other suitable programming language. In effect, the algorithms together with suitable thresholds or other criteria for optimal breast positioning are then programmed in a computer software module, executable by a computer or processor system. Conventional processor technologies, including also PLC (Programmable Logic Controller) technologies, may be used for implementation.

Preferably, the invention is capable of handling retaking of several images, constantly updating which image or image pair (one CC mammogram and one MLO mammogram of each breast) that is considered the most adequate.

The result of the quality assessments may include values of all measurements as well as evaluation of each of the different important areas on the breast that are inspected.

Preferably, the number of quality assessment parameters to be displayed can be configured by the user to allow a selectable level of detail. In the same way, one or more of the threshold values for poor contra good image positioning can normally be configured by the user. For example, referring to [2] and [3] it is recommended that the difference between the CC and MLO posterior nipple lines does not exceed 15 millimeters. The vertical length of the pectoral muscle is another typical configurable parameter. Preferably the MLO image should include the pectoral muscle projecting obliquely across the posterior parts of the image and extending down to the level of the nipple or below. This criterion is easily transformed into real numbers once the coordinate of the nipple is found together with the position where the pectoral muscle reaches the image edge. The quotient of these two values gives a suitable configurable parameter, which can set the level of acceptance concerning the amount of pectoral muscle inclusion on the image. One can also decide whether the nipple needs to be tangentially projected on both the CC and MLO mammograms (when using standard screening projections). In most cases it is enough if the nipple is seen projected tangentially on one image. There are several other factors that can be taken into consideration, like image symmetry (symmetrical at mirroring), exclusion of folds and so forth.

In case of inadequate positioning the result preferably comprises a visual part, using for example a graphical user interface, where inadequate areas are highlighted or otherwise marked and easily spotted. The complete result is preferably saved together with the image, and the result can be accessed and restored whenever desired. Examples of visual indication of inadequately positioned parts of the breast are illustrated schematically in FIGS. 5A and 5B, in which a simple outline of geometric objects are provided as an overlay on top of the mammogram images around the areas of concern.

Finally, a natural extension of the invention is to make it applicable on mammograms of other projections besides the standard screening projections: CC and MLO, which were specifically mentioned in the example of FIGS. 4A and 4B. For example, the LM and ML projections, which are used within clinical mammography, would also be suitable.

The invention is not limited to the exemplary embodiments described above. Further improvements, modifications and changes, which retain the basic underlying principles disclosed and claimed herein, are within the scope of the invention.

### REFERENCES

[1] U.S. Pat. No. 5,572,565.

[2] The art of mammographic positioning; Radiology; Eklund, G. W, Cardenosa; 1992, 30:1, 21-53.

[3] Assessing adequacy of mammographic image quality; Radiology; Eklund, G. W, Cardenosa; 1994, 190:297-307.

[4] European guidelines for quality assurance in mammography screening, third edition; European Commission.

[5] Kvalitetssäkrad bildtagning vid mammografiscreening (Quality secured imaging at Mammography Screening. Freely translated); Läkartidningen; Edström, C, Ståhl, M, 98:10, 1097-1101.

1-30. (canceled)

31. A method for mammographic image quality assurance comprising computerized processing of at least one digital mammographic image to identify landmark areas of the breast, characterized in that said method further comprises:
computerized processing based on image processing measurements on identified landmark areas of the breast together with at least one associated criterion for assessing image positioning quality to produce an automated real-time positioning quality assessment result; and
communicating said positioning quality assessment result on a user interface to enable a real-time decision by a technologist whether to retake said at least one mammographic image with improved positioning.

32. The method according to claim 31, wherein said at least one criterion for assessing image positioning quality includes a set of threshold values.

33. The method according to claim 31, further comprising the step of retaking said at least one mammographic image with improved positioning if the quality assessment result indicates that the image is inadequately positioned.

34. The method according to claim 33, comprising multiple retakes of inadequately positioned images, continuously updating which image or set of images among said multiple retakes that is considered most adequate.

35. The method according to claim 31, wherein said positioning quality assessment result further comprises visual indication of inadequately positioned areas of the breast on a graphical user interface.

36. The method according to claim 31, wherein said positioning quality assessment result includes a number of user-configurable parameters, thus allowing a selectable level of detail in communicating the result.

37. The method according to claim 31, wherein said positioning quality assessment result includes at least one of a visual part and a statistics part.

38. The method according to claim 31, wherein at least one threshold for poor contra good image positioning in said processing is configurable via a user interface.

39. The method according to claim 31, wherein said at least one mammographic examination comprises several projections.

40. The method according to claim 31, wherein said at least one mammographic examination comprises at least one of a CC (Cranio-caudal) projection, an MLO (Medio-lateral oblique) projection, an LM (Latero-medial) projection and an ML (Medio-lateral) projection.

41. The method according to claim 31, wherein said method is applied in mammographic screening.

42. A system for mammographic image quality assurance, wherein said system comprises means for computerized processing of at least one digital mammographic image to identify landmark areas of the breast, characterized in that said system further comprises:
means for computerized processing based on image processing measurements on identified landmark areas of the breast together with at least one associated criterion for assessing image positioning quality to produce an automated real-time positioning quality assessment result; and
means for communicating said positioning quality assessment result on a user interface to enable a real-time decision by a technologist whether to retake said at least one mammographic image with improved positioning.

43. The system according to claim 42, wherein said at least one criterion for assessing image positioning quality includes a set of threshold values.

44. The system according to claim 42, further comprising means for retaking said at least one mammographic image with improved positioning if the quality assessment result indicates that the image is inadequately positioned.

45. The system according to claim 44, comprising means for multiple retakes of inadequately positioned images, and means for updating which image or set of images among said multiple retakes that is considered most adequate.

46. The system according to claim 42, wherein said positioning quality assessment result includes visual indication of inadequately positioned parts of the breast on a graphical user interface.

47. The system according to claim 42, further comprising means for user-configuration of at least one threshold for poor contra good image positioning in said processing.

48. The system according to claim 42, wherein said system is part of a mammographic screening system.

49. A digital mammography system comprising:
means for generating at least one digital mammographic image of a patient;
means for computerized processing of at least one digital mammographic image to identify landmark areas of the breast,
characterized in that said system further comprises:
means for computerized processing based on image processing measurements on identified landmark areas of the breast together with at least one associated criterion for assessing image positioning quality to produce an automated real-time positioning quality assessment result;
means for determining, based on the positioning quality assessment result, whether the image positioning quality of said at least one mammographic image is sufficient; and
means for retaking, if the image positioning quality is insufficient, said at least one mammographic image with improved positioning.

50. The digital mammography system according to claim 49, further comprising means for communicating said positioning quality assessment result to a technologist.

51. The digital mammography system according to claim 49, wherein said at least one criterion for assessing image positioning quality includes a set of threshold values.

52. A computer program product for performing, when running on a computer, mammographic image quality assessment, said computer program product comprising program means for computerized processing of at least one digital mammographic image to identify landmark areas of the breast, characterized in that said computer program product further comprises:
program means for computerized processing based on image processing measurements on identified landmark areas of the breast together with at least one associated criterion for assessing image positioning quality to produce an automated real-time positioning quality assessment result;
program means for communicating said positioning quality assessment result on a user interface to enable a real-time decision whether to retake said at least one mammographic image with improved positioning while the patient is still present.

53. The computer program product according to claim 52, wherein said at least one criterion for assessing image positioning quality includes a set of threshold values.

54. The computer program product according to claim 52, wherein said program means for communicating said positioning quality assessment result includes program means for enabling visual indication of inadequately positioned parts of the breast on a graphical user interface.

55. The computer program product according to claim 52, further comprising program means for user-configuration of at least one threshold for poor contra good image positioning in said processing.

56. The computer program product according to claim 52, wherein said computer program product is implemented in an image acquisition workstation of a digital mammography system.

57. The computer program product according to claim 56, wherein said computer program product is implemented for integrated operation with existing software in said workstation.

58. The computer program product according to claim 52, wherein said computer program product is carried on a computer-readable medium.
